(19) United States
(12) Patent Application Publication
(10) Pub. No.: US 2022/0267837 A1
(43) Pub. Date: Aug. 25, 2022

(54) METHODS FOR IDENTIFYING CARRIER STATUS AND ASSESSING RISK FOR SPINAL MUSCULAR ATROPHY

(71) Applicant: MYRIAD WOMEN'S HEALTH, INC., South San Francisco, CA (US)

(72) Inventor: Daniel Edmund DAVISON, South San Francisco, CA (US)

(73) Assignee: MYRIAD WOMEN'S HEALTH, INC., South San Francisco, CA (US)

(21) Appl. No.: 17/694,443

(22) Filed: Mar. 14, 2022

Related U.S. Application Data
(63) Continuation of application No. 16/912,597, filed on Jun. 25, 2020, now abandoned.
Application No. 16/912,597 is a continuation of application No. PCT/US2018/068237, filed on Dec. 31, 2018.
(60) Provisional application No. 62/612,625, filed on Dec. 31, 2017.

(51) Int. Cl. C12Q 1/6827

(52) U.S. Cl. CPC C12Q 1/6827; C12Q 2600/156

(57) ABSTRACT

Disclosed is a method of determining whether a human subject is not a carrier of spinal muscular atrophy (SMA). This method includes the steps of (i) collecting a genomic deoxyribonucleic acid (DNA) sample from a human subject; (ii) screening the genomic DNA sample to determine the human subject's copy number of survival of motor neuron 1 (SMN1) gene and whether one of the copies of the SMN1 gene is positive for a polymorphism associated with non-carriers of SMA having two copies of the SMN1 gene; and (iii) determining the human subject as not a carrier of SMA if the human subject includes two copies of the SMN1 gene with one of those copies being positive for the polymorphism. Also disclosed is a method of determining whether an individual has a decreased risk of being a carrier of spinal muscular atrophy (SMA), where the individual is identified to have a decreased risk of being a carrier of SMA when the individual has two copies of the SMN1 gene with one of those copies being positive for the polymorphism.

### CROSS-REFERENCE TO RELATED APPLICATIONS

This application is a continuation of U.S. patent application Ser. No. 16/912,597, filed Jun. 25, 2020, which is a continuation of International Patent Application No. PCT/US2018/068237, filed Dec. 31, 2018, which claims priority benefit to U.S. Provisional Application No. 62/612,625, filed on Dec. 31, 2017, the contents of each of which are hereby incorporated by reference in their entirety for all purposes.

### TECHNICAL FIELD

The present disclosure provides, inter alia, methods for identifying carrier and non-carrier status, as well as for assessing risk for spinal muscular atrophy.

### BACKGROUND

Spinal muscular atrophy (SMA) is a severe neuromuscular disease that is the second most common fatal autosomal recessive disorder after cystic fibrosis (Sugarman et al., “Pan-ethnic carrier screening and prenatal diagnosis for spinal muscular atrophy,” Eur J Hum Genet 20(1):27-32 (2012)). SMA is a genetic disease that results from a low copy number of the survival of motor neuron 1 (SMN1) gene in the genome of an individual in comparison to the broader population. SMA is characterized by degeneration of alpha motor neurons in the spinal cord, which results in progressive proximal muscle weakness and paralysis. SMA has an estimated prevalence of 1 in 10,000 live births and an estimated average carrier frequency of 1/40- 1/60. The homozygous absence of SMN1 gene exon 7 is found in approximately 95% of affected patients. SMA is traditionally categorized into various types. For children with SMA, SMA is categorized as: type I, severe; type II, intermediate; and type III, mild. For adults with mild symptoms of SMA, SMA is categorized as type IV. Additionally, for prenatal onset of very severe symptoms of SMA and early neonatal death due to SMA, SMA is categorized as type 0.

The most common SMA carrier alleles involve a deletion in SMN1 (Sugarman 2012). However, duplication alleles also exist, and common methods of testing (e.g., quantitative PCR) measure total dosage and are therefore unable to distinguish a 2±0 carrier from a 1-1 non-carrier. Luo et al. describe a tag SNP g.27134T>G found to be in linkage with the duplication allele (Luo et al., “An Ashkenazi Jewish SMN1 haplotype specific to duplication alleles improves pan-ethnic carrier screening for spinal muscular atrophy,” Genet Med 16(2):149-156 (2014) (referred to herein as the “Luo Study” or the “Luo Paper”)). Following the recommendations of the Luo Study, clinical diagnostic laboratories have begun to use the presence of this tag SNP in 2-copy individuals as diagnostic of SMA carrier status in Ashkenazi Jewish (AJ) and Asian populations. However, despite the data reported and conclusions made in the Luo Study, it is important to ensure that any clinical diagnostic tests that are based on the tag SNP in 2-copy individuals are indeed accurate in identifying carriers and silent carriers of SMA.

There remains a need for accurate diagnostic screening tests to identify SMA carrier status and for assessing risk for SMA. Such diagnostic tools are valuable for individuals planning to have children.

The present invention is directed to overcoming these and other deficiencies in the art.

### SUMMARY

The present disclosure provides, inter alia, methods for identifying carrier status and assessing risk for spinal muscular atrophy (SMA) in a human subject. More specifically, the present disclosure provides methods that involve using the presence of a polymorphism, such as a single-nucleotide polymorphism (SNP), of an SMA gene and the copy number of that gene to determine the carrier status of an individual for SMA, and to provide an improved risk determination with respect to the disease.

In one aspect, the present disclosure provides a method of determining whether a human subject is not a carrier of SMA. In one embodiment, this method includes the steps of: (i) collecting a genomic deoxyribonucleic acid (DNA) sample from a human subject; (ii) screening the genomic DNA sample to determine the human subject's copy number of survival of motor neuron 1 (SMN1) gene and whether one of the copies of the SMN1 gene is positive for a polymorphism associated with non-carriers of SMA having two copies of the SMN1 gene; and (iii) determining the human subject as not a carrier of SMA if the human subject includes two copies of the SMN1 gene with one of those copies being positive for the polymorphism.

In another aspect, the present disclosure provides a method of determining whether an individual has a decreased risk of being a carrier of SMA. In one embodiment, this method includes the steps of: (i) screening a genomic DNA sample of an individual to determine the individual's copy number of the SMN1 gene and whether one of the copies of the SMN1 gene is positive for a polymorphism associated with non-carriers of SMA who have at least two copies of the SMN1 gene; and (ii) determining the individual to have a decreased risk of being a carrier of SMA if the screening of the genomic DNA sample identifies two copies of the SMN1 gene with one of those copies being positive for the polymorphism.

The present disclosure provides methods that are important in providing accurate diagnostic genetic screening tests to identify SMA carrier status and for assessing risk for SMA, as such diagnostic tools are valuable for individuals planning to have children. Furthermore, the methods of the present disclosure are important to ensure that any clinical diagnostic tests that are based on polymorphisms in 2-copy individuals are indeed accurate in identifying non-carriers, carriers, and silent carriers of SMA.

Other objects, features and advantages of the present invention will become apparent from the following detailed description. It should be understood, however, that the detailed description and specific examples, while indicating preferred embodiments of the invention, are given by way of illustration only, since various changes and modifications within the scope and spirit of the invention will become apparent to one skilled in the art from this detailed description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The accompanying drawings illustrate a number of exemplary embodiments and are a part of the specification. Together with the following description, these drawings demonstrate and explain various principles of the instant disclosure.

The file of this patent contains at least one drawing in color. Copies of this patent or patent publication with color drawing(s) will be provided by the Office upon request and payment of the necessary fee.

FIG. 1 is a table comparing raw count data of SMN1 copy number and tag SNP genotype from the prior Luo Study and the study presented in current Example 1. Within each population, the counts are divided into distinct SMN1 copy number categories (rows labeled 1, 2, 3, 4). Within each such copy number category, the counts are divided into 3 columns according to the tag SNP genotype (AA=negative, Aa=heterozygous positive, aa=homozygous positive).

FIG. 2 is a diagram showing the histograms of the posterior distribution of Ashkenazi Jewish residual risk of being an SMA carrier after testing positive for g.27134T>G from the Luo Study and the current study of Example 1.

FIG. 3 is a table showing risk (1-in-X) of an individual from various populations being an SMA carrier given SNP positive 2-copy genotype. The table includes data taken from the Luo Study and from current Example 1.

FIGS. 4A-4F are graphs illustrating global and local genetic ancestry of various hypothetical populations. FIG. 4A is a graph of Ancestry Component 5× Ancestry Component 3. FIG. 4B is a graph of Ancestry Component 6× Ancestry Component 3. FIG. 4C is a graph of Ancestry Component 6× Ancestry Component 5. FIG. 4D is a graph of Ancestry Component 2′×Ancestry Component 1′. FIG. 4E is a graph of Ancestry Component 3′× Ancestry Component 1′. FIG. 4F is a graph of Ancestry Component 3′× Ancestry Component 2′.

FIG. 5 is a graph of the results of a power simulation study related to SMA carrier determinations and SMA risk assessment. The X-axis is the number of trios in a hypothetical trio study, and the Y-axis is the probability of rejecting the null hypothesis, as discussed in more detail herein.

FIG. 6 is a graph showing the basis for the estimate of 0.00372 as being the posterior mean P(SNP alt allele|1 copy haplotype), as discussed in more detail herein.

FIG. 7 is a schematic drawing depicting the most probable trio genotype configuration under the null hypothesis P(SNP|1-copy haplotype)=0, as discussed in more detail herein.

FIG. 8 is a schematic drawing depicting the most probable trio genotype configuration under the alternate hypothesis P(SNP|1-copy haplotype)>0, as discussed in more detail herein.

Throughout the drawings, identical reference characters and descriptions indicate similar, but not necessarily identical, elements. While the exemplary embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION OF EXEMPLARY EMBODIMENTS

The instant disclosure is directed to, inter alia, methods of excluding an individual from being a carrier or silent carrier of spinal muscular atrophy (SMA) and methods for providing genetic counseling to an individual as to their risk of being a carrier of SMA.

The present disclosure will now be described in detail by way of reference to certain definitions and examples.

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this invention. Although any methods and materials similar or equivalent to those described herein can be used in the practice or testing of the present invention, the preferred methods and materials are described. Practitioners are particularly directed to Sambrook et al., 1989, and Ausubel F M et al., 1993, for definitions and terms of the art. It is to be understood that this invention is not limited to the particular methodology, protocols, and reagents described, as these may vary.

Numeric ranges are inclusive of the numbers defining the range. The term about is used herein to mean plus or minus up to ten percent (10%) of a value. For example, “about 100” refers to any number between 90 and 110.

Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention, which can be had by reference to the specification as a whole. Accordingly, the terms defined immediately below are more fully defined by reference to the specification as a whole.

As discussed herein, the present disclosure makes reference to the findings described in Luo et at, “An Ashkenazi Jewish SMN1 haplotype specific to duplication alleles improves pan-ethnic carrier screening for spinal muscular atrophy,” Genet Med 16(2):149-156 (2014) (referred to herein as the “Luo Study” or the “Luo Paper”). The findings described in the Luo Paper were subsequently included in the specification of U.S. Pat. No. 9,994,898 to Edelmann et al., issued Jun. 12, 2018, and titled MATERIALS AND METHODS FOR IDENTIFYING SPINAL MUSCULAR ATROPHY CARRIERS. While the data presented in Examples 1 and 2 of the present disclosure call into question the conclusions made in the Luo Paper and U.S. Pat. No. 9,994,898, there are a number of terms, definitions, and techniques described in these documents that are relevant in describing the methods of the present disclosure.

Therefore, to assist in further understanding the technology associated with the methods of the present disclosure, provided below are various definitions to facilitate an unambiguous disclosure of the various aspects of the disclosure, some of which are contained in U.S. Pat. No. 9,994,898.

As used herein, a “polymorphism” is a difference in DNA or RNA sequence among individuals, groups, or populations that gives rise to different alleles. The alleles may be alleles of a gene encoding a gene product, such as SMN1, and the polymorphism may involve a sequence change (relative to wild type sequence) in the coding region, in the transcribed but untranslated region associated with a gene, in the expression control region of a gene, in the proximal nucleic acid environment of a gene or located at some distance from the gene. Typically, polymorphisms of interest in identifying SMA carriers will be genetically linked to the SMN1 gene. Exemplary polymorphisms include substitutions of one or more (e.g., 2, 3, 4, 5, 6, 7, 8, 9, 10, or more) nucleotides, deletions of a polynucleotide region comprising at least 1, 2, 3, 4, 5, 6, 7, 8, 9, 10, 15, 20, 25, 30, 40, 50, 75, 100, 150, 200, 250, 500, 1,000, or more nucleotides, and insertions of nucleotides into a polynucleotide region wherein the insertion is of a length defined above in the context of addressing deletions.

As used herein, a “SNP” is a single-nucleotide polymorphism, or single nucleotide difference in the nucleic acid sequence relative to the wild type sequence. As provided herein, the presence of the g.27134T>G SNP in an individual's genomic DNA can be used in these methods.

As used herein, a “haplotype” is a partial genotype of at least one determinant containing at least one polymorphism, such as single-nucleotide polymorphism (SNP), a deletion or an insertion, on a chromosome. For haplotypes comprising more than one polymorphism, the individual polymorphisms exhibit statistically significant linkage disequilibrium. Exemplary polymorphisms are single- or multiple-nucleotide substitutions, insertions or deletions and each polymorphism may be localized to a determinant that is a gene recognized in the art, such as SMN1, a new gene, or an extragenic region of a chromosome.

As used herein, “Copy number” refers to the number of physical copies of a genetic determinant, such as a gene, or region of the genome of an organism.

As used herein, a “carrier” or genetic “carrier” is an individual containing at least one copy of an allele of a genetic determinant that is involved in elaborating a given phenotype, such as SMA, provided that the individual containing the copy or copies of the determinant does not exhibit the phenotype.

As used herein, a “silent carrier” is a carrier that cannot be detected using a copy number-based diagnostic technique conventional in the art.

As discussed herein, the present disclosure provides methods of excluding an individual from being a carrier or silent carrier of spinal muscular atrophy (SMA) and methods for providing genetic counseling to an individual as to their risk of being a carrier of SMA.

SMA is caused by mutations in the survival motor neuron (SMN) gene. The SMN gene includes nine exons and gives rise to the 38-kD SMN protein. The SMN protein plays a critical role in assembly and regeneration of small nuclear ribonuclear proteins. The SMN protein also functions in axonal RNA transport and mRNA splicing. The SMN gene is located in an inverted, duplicated region of chromosome five, which includes two highly homologous copies of the SMN gene, namely SMN1, which is a telomeric copy of the gene, and SMN2, which is a centromeric copy.

A single point mutation in exon 7 (C>T at position 840) distinguishes the SMN1 gene from the SMN2 gene, which contains the point mutation in exon 7. This point mutation affects splicing, so most transcripts arising from the SMN2 gene lack exon 7. The SMN1 gene transcribes full-length mRNA, while the SMN2 gene primarily transcribes a shortened mRNA species lacking exon 7. Because SMN protein without exon 7 has a lower oligomerization efficiency, it is much more prone to degradation. Hence, the point mutation in exon 7 of the SMN2 gene results in lower overall generation of the SMN protein from the SMN2 gene in comparison to the SMN1 gene. Ninety-five percent of SMA affected individuals have a homozygous deletion involving the SMN1 gene. Although affected individuals retain at least one copy of SMN2, the SMN2 gene only partially compensates for the homozygous loss of the SMN1 gene due to the lower oligomerization efficiency of the SMN2 gene.

As described in International Application No. PCT/US2018/014163 to Counsyl, Inc. (now Myriad Women's Health, Inc.), filed Jan. 18, 2018 and titled SYSTEMS AND METHODS FOR QUANTITATIVELY DETERMINING GENE COPY NUMBER, there are systems and methods that allow for highly reliable real-time determination of the SMN1 gene and/or SMN2 gene in the genome of an individual. As described in greater detail below, the copy number of the target SMN1 gene and/or SMN2 gene may be determined using a multiplex real-time PCR (qPCR) procedure in which reference genes are amplified in the same reaction mixtures as the target genes. The accuracy of the copy number determination may be further increased through the use of analytical modeling of the qPCR results, allowing for robust copy number determinations using minimal sample amounts and few sample replicates. While the exemplary systems and methods are directed to determining SMN1 and/or SMN2 gene copy numbers in an individual's genomic DNA, the systems and methods described in PCT/US2018/014163, and incorporated herein, are not limited to these genes and may be utilized in the determination of copy numbers of any suitable genes, without limitation.

In certain embodiments described in PCT/US2018/014163, and as incorporated herein, the systems and methods may allow for determination of SMN1 gene copy numbers for genomic DNA samples in order to screen for various types of SMA. The systems and methods may be used to screen for SMA types I, II, III, and IV, which are shown in Table 1 below.

TABLE 1
Spinal Muscular Atrophy Basic Information

| Disease | Spinal Muscular Atrophy (SMA) | |
|---|---|---|
| SMA MIM IDs | 253300 (TYPE I; SMA1); | |
| | 253400 (TYPE III; SMA3); | |
| | 253550 (TYPE II; SMA2); | |
| | 271150 (TYPE IV; SMA4) | |
| SMA gene | SMN1 | |
| SMN1 MIM ID | 600354 | |
| SMN1 gene map locus | 5q12.2-q13.3 | |
| SMN1 wildtype copy number | ≥2 | copies |
| SMN1 carrier copy number | 1 | copy |
| SMN1 affected copy number | 0 | copies |

In one embodiment described in PCT/US2018/014163, extracted genomic DNA may be subjected to qPCR to quantify the copy number of the SMN1 exon 7 at the +6 nucleotide that distinguishes the SMN1 gene from the SMN2 gene. A multiplex qPCR utilizing target gene and reference gene assays may be performed on genomic DNA samples, with the reference gene assays targeting endogenous housekeeping genes having an invariant copy number. The amount of SMN1 gene in a sample may be measured relative to the reference gene corresponding to each respective reference gene assay.

As described in PCT/US2018/014163, individuals having genomic DNA samples that are determined to have half the quantity of SMN1 genes relative to the endogenous reference genes (i.e., genomic DNA samples having an SMN1 gene copy number of 1 in comparison to a reference gene copy number of 2) may be identified as SMA carriers. For example, a diploid genome of an SMA carrier includes a single copy of the SMN1 gene and 2 copies of an endogenous reference gene, such as the hTERT gene. Accordingly, simultaneous amplification of the SMN1 gene and the hTERT gene through qPCR of a genetic DNA sample of an SMA carrier may result in double the amount of the hTERT gene in comparison to the SMN1 gene. Alternatively, individuals having genomic DNA samples that are determined to have an SMN1 copy number of 2 or 3 may be identified as SMA non-carriers. For example, a diploid genome of an SMA non-carrier may include 2 copies of the SMN1 gene and 2 copies of the hTERT gene. Simultaneous amplification of the SMN1 gene and the hTERT gene through qPCR of a genetic DNA sample of an SMA non-carrier having an SMN1 gene copy number of 2 may result in approximately the same amount of the hTERT gene and the SMN1 gene.

In one aspect, the present disclosure is directed to a method of determining whether a human subject is not a carrier of SMA. This method includes the steps of: (i) collecting a genomic DNA sample from a human subject; (ii) screening the genomic DNA sample to determine the human subject's copy number of the SMN1 gene and whether one of the copies of the SMN1 gene is positive for a polymorphism associated with non-carriers of SMA having two copies of the SMN1 gene; and (iii) determining the human subject as not a carrier of SMA if the human subject includes two copies of the SMN1 gene with one of those copies being positive for the polymorphism.

In accordance with this method, the step of collecting a genomic DNA sample from a human subject can be performed using all or portions of the methods, techniques, and materials that are conventional in the relevant art. Similarly, methods, techniques, and materials that are conventional in the relevant art can be employed to perform all or portions of the step of screening the genomic DNA sample to determine the human subject's copy number of the SMN1 gene and whether one of the copies of the SMN1 gene is positive for a polymorphism associated with non-carriers of SMA having two copies of the SMN1 gene. Furthermore, methods, techniques, and materials that are conventional in the relevant art can be employed to perform all or portions of the step of determining the human subject as not a carrier of SMA if the human subject includes two copies of the SMN1 gene with one of those copies being positive for the polymorphism. Exemplary disclosures of such methods, techniques, and materials referenced in this paragraph can be found in, without limitation, US-2016/0188793A1, US-2014/0199695A1, US-2016/0153037A1, US-2016/0103959A1, US-2015/0111203A1, US-2018/0129778A1, WO 2017/156290A1, WO 2018/112249A1, WO 2018/117986A1, WO 2018/144228A1, WO 2018/170443A1, WO 2017/165463A1, PCT/US2018/014173, U.S. Pat. Nos. 7,424,368, and 9,994,898.

In one embodiment of this method, the polymorphism is a single-nucleotide polymorphism (SNP) in intron 7 of the SMN1 gene. In another embodiment, the SNP is g.27134T>G.

In accordance with this method, determining the human subject as not a carrier of SMA includes identifying the human subject to have one copy of the SMN1 gene on each of the human subject's two 5q13.2 chromosomes, along with one of the SMN1 genes also being positive for the g.27134T>G SNP.

In one embodiment, this method further involves providing the human subject a risk assessment of being a non-carrier (1+1), carrier (1+0), or silent carrier (2+0) of SMA based on the copy number of the SMN1 gene and the presence or absence of the g.27134T>G SNP on one of the SMN1 genes.

In another aspect, the present disclosure is directed to a method of determining whether an individual has a decreased risk of being a carrier of SMA. This method includes the steps of: (i) screening a genomic DNA sample of an individual to determine the individual's copy number of the SMN1 gene and whether one of the copies of the SMN1 gene is positive for a polymorphism associated with non-carriers of SMA who have at least two copies of the SMN1 gene; and (ii) determining the individual to have a decreased risk of being a carrier of SMA if the screening of the genomic DNA sample identifies two copies of the SMN1 gene with one of those copies being positive for the polymorphism.

In accordance with this method, techniques, methods, and materials that are conventional in the relevant art can be employed to perform all or portions of the step of screening a genomic DNA sample of an individual to determine the individual's copy number of the SMN1 gene and whether one of the copies of the SMN1 gene is positive for a polymorphism associated with non-carriers of SMA who have at least two copies of the SMN1 gene. Furthermore, methods, techniques, and materials that are conventional in the relevant art can be employed to perform all or portions of the step of determining the individual to have a decreased risk of being a carrier of SMA if the screening of the genomic DNA sample identifies two copies of the SMN1 gene with one of those copies being positive for the polymorphism. Exemplary disclosures of such methods, techniques, and materials referenced in this paragraph can be found in, without limitation, US-2016/0188793A1, US-2014/0199695A1, US-2016/0153037A1, US-2016/0103959A1, US-2015/0111203A1, US-2018/0129778A1, WO 2017/156290A1, WO 2018/112249A1, WO 2018/117986A1, WO 2018/144228A1, WO 2018/170443A1, WO 2017/165463A1, PCT/US2018/014173, U.S. Pat. Nos. 7,424,368, and 9,994,898.

In one embodiment of this method, the polymorphism is a SNP in intron 7 of the SMN1 gene. In another embodiment, the SNP is g.27134T>G.

In accordance with this method, determining the individual to have a decreased risk of being a carrier of SMA includes identifying the individual as having one copy of the SMN1 gene on each of the individual's two 5q13.2 chromosomes, along with one of the SMN1 genes also being positive for the g.27134T>G SNP.

In one embodiment, this method further involves counseling the individual of the individual's decreased risk of being a carrier (1+0) or silent carrier (2+0) of SMA based on the individual's copy number of the SMN1 gene and the presence or absence of the g.27134T>G SNP on one of the SMN1 genes. More specifically, if the individual has two copies of the SMN1 gene along with one of the SMN1 genes being positive for the g.27134T>G SNP, the individual has a decreased risk of being an SMA carrier. In such a case, the individual is more likely to have one copy of the SMN1 gene on each of the individual's two 5q13.2 chromosomes, rather than having two copies of the SMN1 gene on a single 5q13.2 chromosome.

In one embodiment, this method further involves collecting the genomic DNA sample from the individual prior to the screening step.

### EXAMPLES

The present invention is described in further detail in the following examples which are not in any way intended to limit the scope of the invention as claimed. The attached Figures are meant to be considered as integral parts of the specification and description of the invention. All references cited are herein specifically incorporated by reference for all that is described therein. The following examples are offered to illustrate, but not to limit the claimed invention.

### Example 1

### Duplication Tag SNP g.27134T>G should not be Considered Diagnostic of Spinal Muscular Atrophy Carrier Status

A study was conducted to investigate prior conclusions in the field of the presumed linkage between the tag SNP g.27134T>G with the duplication allele of SMN1, particularly as reported in Luo et al., “An Ashkenazi Jewish SMN1 haplotype specific to duplication alleles improves pan-ethnic carrier screening for spinal muscular atrophy,” Genet. Med., 16(2):149-156 (2014) (referred to herein as the “Luo Study” or “Luo Paper”). This Example 1 describes this study and its conclusions.

### Methods

A full-likelihood Bayesian model was developed for the CN and SNP data and for post-test risk. The model for the CN and SNP count data has the structure of a multinomial model, in which the cell probabilities depend on the (unknown) SNP+copy number haplotype frequencies in a given population.

The residual risk that a CN=2 individual is an SMA carrier conditional on their SNP genotype was calculated by sampling from the posterior distribution of the haplotype frequencies, and using these samples to construct a sample from the posterior distribution of residual risk values. This procedure propagates all uncertainty regarding population allele frequencies and SNP/copy-number correlations through to the final residual risk calculation. Rejection sampling was used to sample from posterior probability distributions that are not analytically tractable.

Sequencing was performed of 12,089 individuals, randomly selected without regard to ethnicity or SMA genotype/phenotype, to determine SMN1 CN and g.27134T>G SNP genotype. Posterior residual risk values were computed for the original Luo Study data set, and for the new data set of the current study.

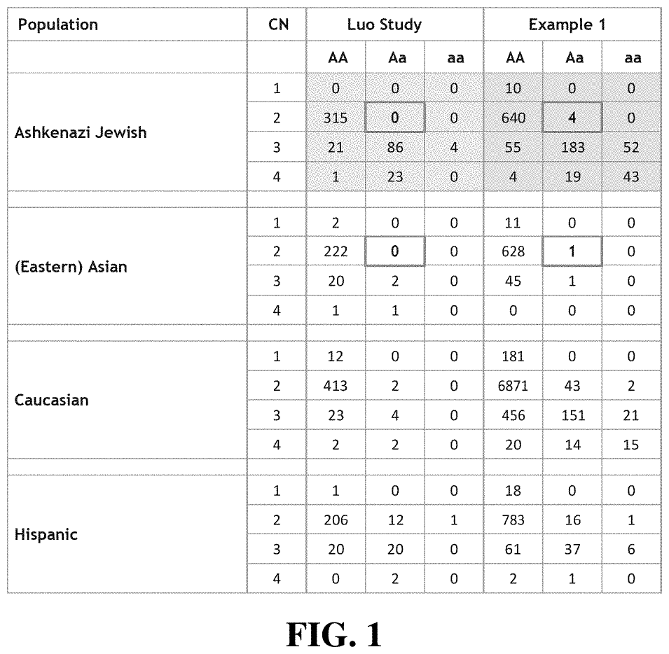

As shown in the table of FIG. 1, raw count data was compared of SMN1 copy number and tag SNP genotype from the prior Luo Study and the study presented in current Example 1.

### Results

Estimating the posterior distribution over model parameters shows that maximum likelihood/least-squares estimation (ML/LS) can lead to erroneous calculation of residual risk, with ML/LS estimates being either substantially too high or too low. For example, although ML/LS residual risk for AJs in the Luo Study data is 100% (1 in 1), the posterior mean value of this risk is much lower: 1 in 2.9. Similarly, for Asian individuals the ML/LS estimate is 100% (1 in 1), whereas the posterior mean is 1 in 7.9.

In contrast to the Luo Study data, we found a small number of 2-copy AJ individuals positive for the tag SNP, whose presence decreases the inferred linkage between the duplication allele and the SNP. These individuals had genetic ancestry typical of self-reported AJ individuals.

A diverse test population demonstrated heterogeneity of tag SNP performance among Asian populations: while self-reported Southeast Asians had statistics similar to the Luo Study “Asian” population, among both East and South Asians the inferred linkage between the tag SNP and the SMN1 duplication is substantially lower, conveying much smaller residual risk.

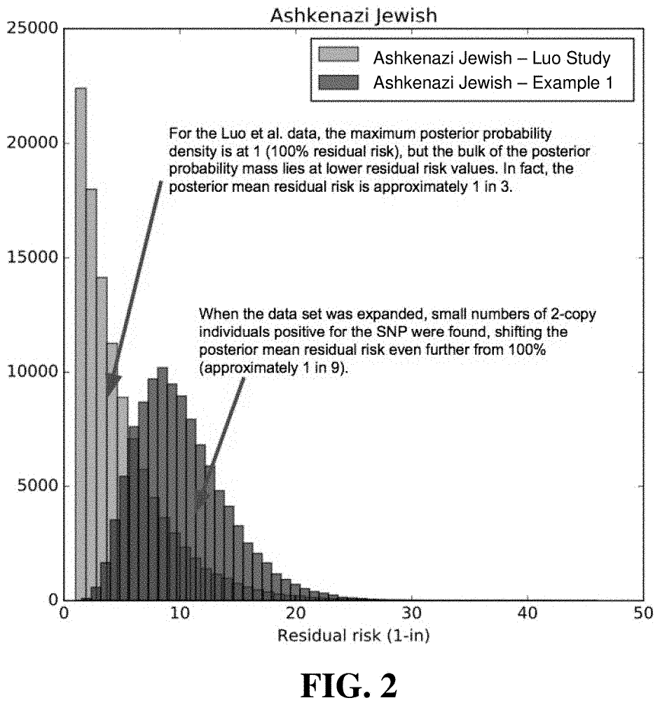

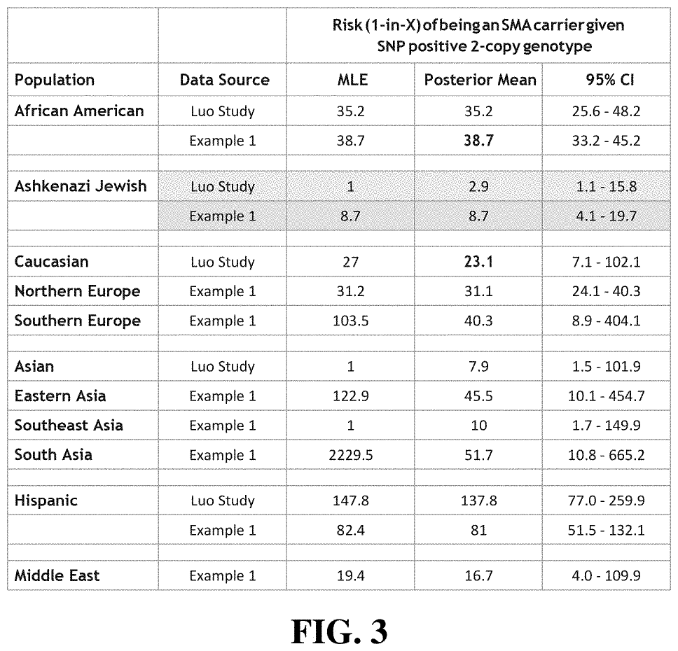

As supported by FIG. 2 and FIG. 3, although the posterior mode is at 100% for the Luo Study data, it is not appropriate to treat the tag SNP as diagnostic for SMA carrier status in the Ashkenazi Jewish population.

FIG. 2 shows histograms of the posterior distribution of Ashkenazi Jewish residual risk of being an SMA carrier after testing positive for g.27134T>G from the Luo Study, and the current study of Example 1. Each histogram comprises 100,000 samples simulated from the posterior distribution of the residual risk. Notice that for the Luo Study, while the probability distribution peaks at a residual risk of 100% (1 in 1), there is substantial probability weight away from this mode. In fact, the posterior mean is 1 in 2.9. The conclusion that the residual risk is substantially lower than 100% is even stronger for the current study of Example 1 (posterior mean residual risk 1 in 8.7).

The table shown in FIG. 3 includes data taken from the Luo Study and from current Example 1, and illustrates the risk (1-in-X) of an individual from various populations being an SMA carrier given SNP positive 2-copy genotype. The populations studied were African American, Ashkenazi Jewish, Caucasian, Northern European, Southern European, Asian, Eastern Asian, Southeast Asian, South Asian, Hispanic, and Middle Eastern.

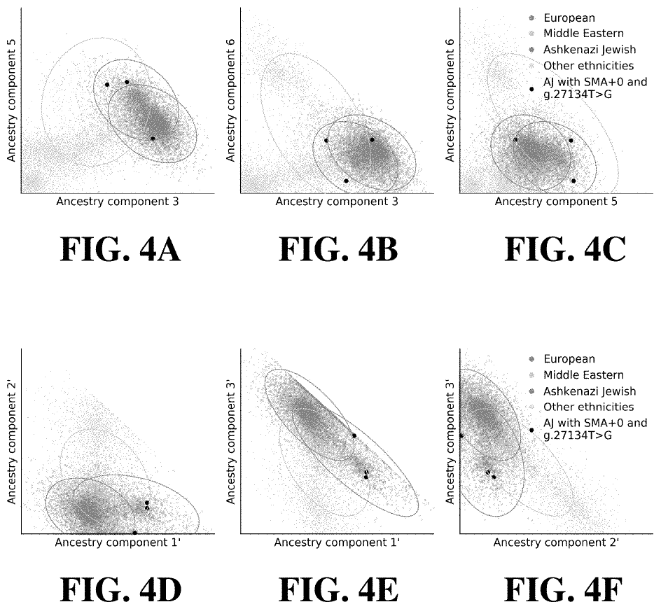

As shown in FIGS. 4A-4F, the 2-copy individuals who self-reported as Ashkenazi Jewish and are positive for the tag SNP are within the normal range of Ashkenazi Jewish genetic ancestry.

FIGS. 4A-4F are graphs illustrating global and local genetic ancestry of various hypothetical populations. FIGS. 4A, 4B, and 4C show global genetic ancestry (amount of ancestry shared with 7 hypothetical ancestral populations defined using reference populations across the world), highlighting 3 individuals who are both 2-copy and positive for the SNP tag (small black circles). Individuals self-reporting as European, Middle Eastern, or Ashkenazi Jewish are also shown. FIGS. 4D, 4E, and 4F show the same cohort, analyzed for local genetic ancestry shared with 3 hypothetical ancestral populations defined using only European, Middle Eastern, and Ashkenazi Jewish reference populations, highlighting the same 3 individuals.

### Conclusions

Uncertainty in population haplotype frequency estimates should be propagated forwards into residual risk calculations using a standard Bayesian probability framework. The Luo Study's conclusions were based on least-squares/maximum likelihood, but using these analysis methods leads to the conclusion in certain populations that a proband testing positive for the tag SNP is certainly a carrier, when in fact it is still more probable that they are not.

Phasing experiments could resolve carrier status in individual cases and would improve understanding of the implications of the tag SNP for carrier status.

### Example 2

### SMA g.27134T>G Trio Simulation Study

As a follow-up to the study presented in current Example 1, disclosed below in Example 2 is a simulation study that sets forth a provisional estimate of how many SMA trios one would need to collect to determine informativeness of the g.27134T>G SNP. The current Example 2 also develops some likelihood calculations that are useful in discussing and analyzing an SMA trio study. The branch is forked off a branch of a separate, internal study that implements calculations for the g.27134T>G SNP instead of copying them from the previous Luo Study, as described in Luo et al., “An Ashkenazi Jewish SMN1 haplotype specific to duplication alleles improves pan-ethnic carrier screening for spinal muscular atrophy,” Genet. Med., 16(2):149-156 (2014) (referred to herein as the “Luo Study” or “Luo Paper”).

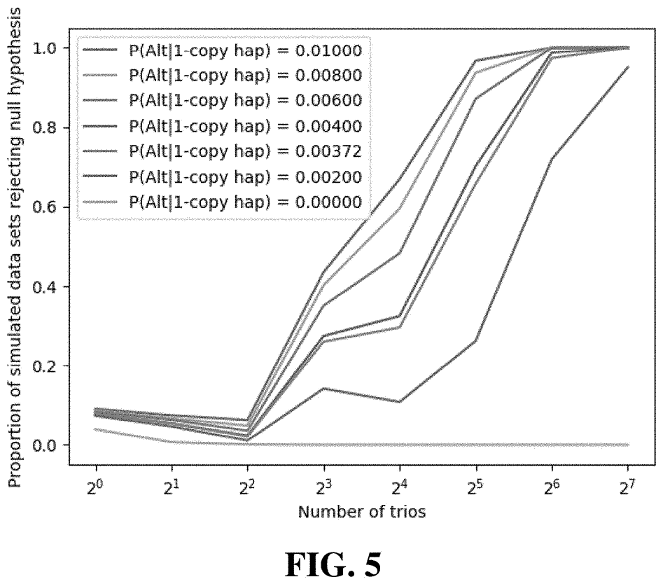

Results of the power simulation study of current Example 2 are shown in the graph of FIG. 5. As shown in FIG. 5, the X axis is the number of trios in a hypothetical trio study, and the Y axis is the probability of rejecting the null hypothesis. Each colored line corresponds to a different hypothesis about the frequency of the SNP alt allele on 1-copy haplotypes. At the bottom (pink) we have the null hypothesis: P(SNP alt alleles 1 copy haplotype)=0. This says that the following is correct: the SNP alt allele only occurs on 2-copy haplotypes (so a 2-copy individual with the SNP is a carrier with 100% probability) (as discussed in the Luo Paper). In the middle (purple) is a parameter value close to what we believe from our previous work describes the Ashkenazi Jewish population: posterior mean P(SNP alt alleles 1 copy haplotype)=0.00372.

Thus, the graph of FIG. 5 shows, for example, that if the parameter estimate is correct, and if we obtain 32 trios, then we have approximately a 60% chance of (correctly) rejecting the null hypothesis (40% chance of a false negative). If, however, we were to obtain 64 trios, then we would be almost certain to (correctly) reject the null hypothesis.

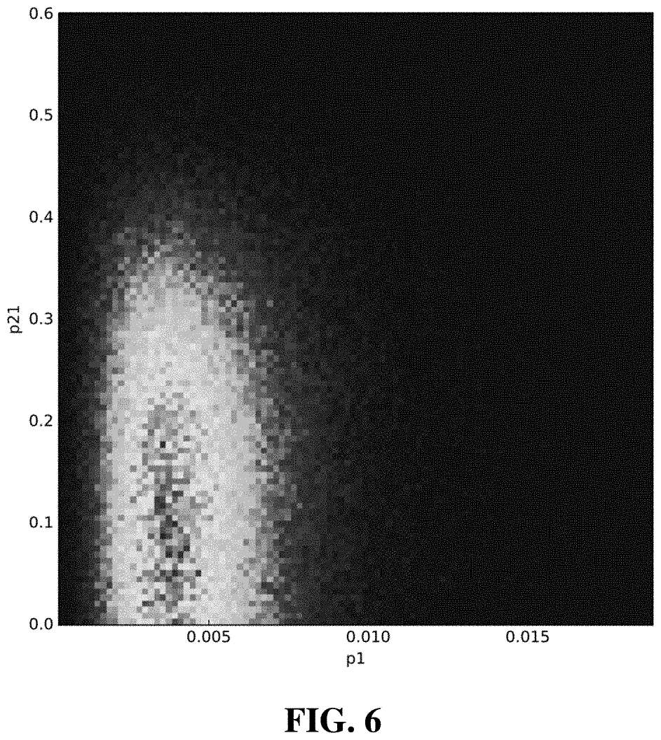

FIG. 6 is a graph of a plot that shows the basis for the estimate of 0.00372 as being the posterior mean P(SNP alt allele|1 copy haplotype). It shows the distribution of samples from the posterior distribution of the parameters, collected via rejection sampling. The parameter in question is on the X axis.

To further illustrate the study and conclusions of current Example 2, recall that for an SMA 2-copy individual, we do not know if they are a carrier or not (both possibilities have substantial probability). g.27134T>G is a SNP that can provide additional information, helping resolve the answer in one direction or the other. The Luo Paper discusses this SNP. In particular, the Luo Paper claims that g.27134T>G can be considered to be 100% diagnostic of carrier status in Ashkenazi Jewish or some Asian populations. However, in accordance with the work relating to the present disclosure of Example 2, the results reported in the Luo Paper are questionable. Specifically, the findings of the present disclosure of Example 2 suggest that, in fact, even in AJ and Asian populations, the posterior probability of being a carrier conditional on being 2-copy and SNP positive is much less than 1. The reason for the disagreement is the Luo Paper's use of a least-squares/frequentist analysis method.

Therefore, the present disclosure addresses the question of how to obtain a more definitive answer. One possibility is to do a trio study in which we find, for example, Ashkenazi Jewish patients that are 2-copy and positive for the SNP, and sequence their parents.

Informally, one can see that a trio study should be informative, as discussed in more detail below.

The present disclosure of Example 2 provides teachings to estimate how many trios one would need to sequence in order to answer the question with suitable certainty.

### The Competing Hypotheses and Some Intuition for the Problem

Null Hypothesis (from the Luo Paper):

The null hypothesis is that: if you are 2-copy and you have the SNP alt allele, then you certainly are a carrier. In other words, you have one chromosome with 2 copies, and another with 0 copies. An equivalent way of stating the null hypothesis is that the frequency of 1-copy chromosomes with the alt allele is zero. This follows because, if it were not zero, then some non-carriers (1+1 configuration) would have the SNP alt allele, and the posterior probability of being a carrier given the alt allele would be less than 1 under Bayes' rule.

Alternate Hypotheses (from Current Example 2):

The alternate hypothesis is that there is a non-zero probability of the SNP alt allele occurring on a 1-copy haplotype. This implies that the posterior probability of being a carrier given the alt allele is less than 1, contra the claims of the Luo Paper.

### Intuition:

The intuition behind the results of the present disclosure requires some further explanation. Recall that our trios always (by design) feature a 2-copy individual (the “child”) with the SNP alt allele (observed genotype 2+). Therefore, all the relevant probability distributions must be conditioned on this ascertainment policy.

Given the observation that a child is 2-copy and has the alt allele, it is not known as to what the underlying child genotype is (silent carrier or not), and it is not known what the parent genotypes are. Thus, one can ask what the posterior probability distribution is over these things, conditional on the child observation.

Let's look at the child and parent genotypes that have highest posterior probability conditional on the child being 2+, under the null and alternate hypotheses.

### Null Hypothesis Discussion:

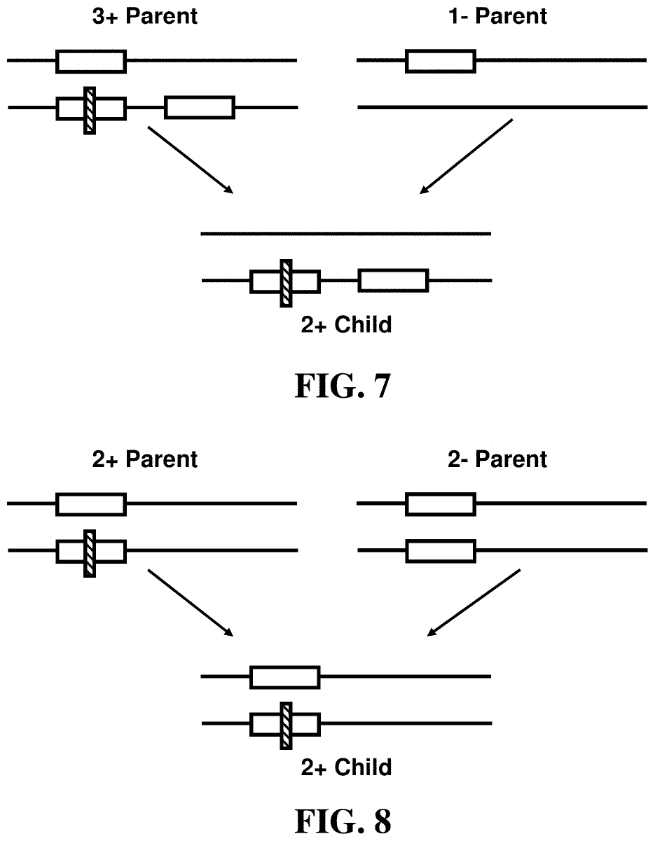

Under the null hypothesis P(SNP|1-copy haplotype)=0, the genotype of the 2-copy SNP positive child must be 2+, 0 since 1-copy haplotypes with the SNP alt allele don't exist. FIG. 7 is a diagram that shows what is by far the most probable trio genotype configuration (83% probability, next most probable has 5% probability); specifically, a 3+ parent, a 1− parent, and a 2+ child. Each blank, rectangular box represents one copy of the SMN1 gene. Each vertical, rectangular box with diagonal filling represents the present of the SNP.

### Alternate Hypothesis Discussion:

Under the alternate hypothesis P(SNP|1-copy haplotype)>0, the alt allele is allowed on a 1-copy background. How frequent this is depends on the parameter value. However, recall that our entire analysis is conditioned on the child having 2-copies and being positive for the SNP, and that we have a strong prior belief that a 2-copy individual has two 1-copy chromosomes. Because of this conditioning, even if the SNP is rather rare on 1-copy haplotypes, it is still the case that the most probable explanation is that the child has one of these rare 1-copy SNP-positive haplotypes. Consistent with this logic, the most probable trio configuration is as shown in the diagram set forth in FIG. 8; specifically, a 2+ parent, a 2− parent, and a 2+ child. Each blank, rectangular box represents one copy of the SMN1 gene. Each vertical, rectangular box with diagonal filling represents the present of the SNP.

Thus, in accordance with the present disclosure of Example 2, when we simulate in regions of parameter space away from the null hypothesis, we tend to quite frequently generate the second type of trio genotype configuration. Since these are extremely improbable under the null hypothesis, such data sets result in rejecting the null hypothesis even when they comprise relatively few trios.

### Modeling and Calculations Behind the Simulation Study:

The parameter space is one-dimensional: it is the frequency of the SNP alt allele on the 1-copy haplotype background, ranging from 0 to 1.

The set of possible trio genotype configurations is finite and reasonably small. We start by computing, for every trio, at every point of a grid of parameter values, the probability of the observed trio data, conditional on the observed child data being 2-copy SNP positive. An expression for this probability is as follows:

Let π=(π₀₋, π₁₋, π₁₊, π₂₋, π₂₊) be the vector of haplotype frequencies. We have two sources of data that we use to estimate π:

(a) Counts of copy-number/SNP calls in historical lab data; and

(b) Family trio data.

Family trio data is collected conditional on the observed genotype of the child.

Therefore, the appropriate likelihood is the probability of the mother and father data, given the child data and the haplotype frequencies:

$$\begin{matrix}p ( mother , father \vert child , π ) = \frac{\begin{matrix}p ( mother \vert π ) p ( father \vert π ) \\ p ( child \vert mother , father , π )\end{matrix}}{p ( child \vert π )} \\ = \frac{\begin{matrix}\sum {G}_{mother} \sum {G}_{father} p ( {G}_{mother} \vert π ) p ( {G}_{father} \vert π ) \\ \sum {G}_{child} p ( {G}_{child} \vert {G}_{mother} , {G}_{father} )\end{matrix}}{{G}_{child} p ( {G}_{child} \vert π )}\end{matrix}$$

In this expression, the summations are understood to be over the set of underlying genotypes consistent with the observed genotype of each individual.

It is important to condition on the child being 2-copy SNP positive since this is the ascertainment that can be used in further studies.

A simulated data set comprises some number T of trios. For a given value of the parameter, we simulate a data set of T trios by treating the probability distribution over trios calculated in the first step as the cell probabilities in a multinomial distribution.

A simulated data set is defined to reject the null hypothesis if the 95% highest posterior density interval for the parameter excludes zero.

The 95% highest posterior density interval is computed as follows: (i) compute the likelihood of the observed data set over a grid of parameter values; (ii) numerically integrate the likelihood surface (interpreted as a posterior, with an implicit flat prior) to form the CDF; and (iii) use numerical optimization to find the 95% interval with highest average density.

The likelihood of a simulated data set comprising T trios given parameters theta is the product of the T individual trio probabilities at parameter value theta.

Other advantages which are obvious and which are inherent to the disclosure will be evident to one skilled in the art. It will be understood that certain features and sub-combinations are of utility and may be employed without reference to other features and sub-combinations. This is contemplated by and is within the scope of the claims. Since many possible embodiments may be made of the disclosure without departing from the scope thereof, it is to be understood that all matter herein set forth or shown in the accompanying drawings is to be interpreted as illustrative and not in a limiting sense.

What is claimed is:

1. A method of determining whether a human subject is not a carrier of spinal muscular atrophy (SMA), said method comprising:
collecting a genomic deoxyribonucleic acid (DNA) sample from a human subject;
screening the genomic DNA sample to determine the human subject's copy number of survival of motor neuron 1 (SMN1) gene and whether one of the copies of the SMN1 gene is positive for a polymorphism associated with non-carriers of SMA having two copies of the SMN1 gene; and
determining the human subject as not a carrier of SMA if the human subject includes two copies of the SMN1 gene with one of those copies being positive for the polymorphism.

2. The method according to claim 1, wherein the polymorphism is a single-nucleotide polymorphism (SNP) in intron 7 of the SMN1 gene.

3. The method according to claim 2, wherein the SNP is g.27134T>G.

4. The method according to claim 1, wherein determining the human subject as not a carrier of SMA includes identifying the human subject to have one copy of the SMN1 gene on each of the human subject's two 5q13.2 chromosomes, with one of the SMN1 genes also being positive for the g.27134T>G SNP.

5. The method according to claim 1 further comprising:
providing the human subject a risk assessment of being a non-carrier (1+1), carrier (1+0), or silent carrier (2+0) of SMA based on the copy number of the SMN1 gene and the presence or absence of the g.27134T>G SNP on one of the SMN1 genes.

6. A method of determining whether an individual has a decreased risk of being a carrier of spinal muscular atrophy (SMA), said method comprising:
screening a genomic DNA sample of an individual to determine the individual's copy number of survival of motor neuron 1 (SMN1) gene and whether one of the copies of the SMN1 gene is positive for a polymorphism associated with non-carriers of SMA who have at least two copies of the SMN1 gene; and
determining the individual to have a decreased risk of being a carrier of SMA if the screening of the genomic DNA sample identifies two copies of the SMN1 gene with one of those copies being positive for the polymorphism.

7. The method according to claim 6, wherein the polymorphism is a single-nucleotide polymorphism (SNP) in intron 7 of the SMN1 gene.

8. The method according to claim 7, wherein the SNP is g.27134T>G.

9. The method according to claim 6, wherein determining the individual to have a decreased risk of being a carrier of SMA includes identifying the individual as having one copy of the SMN1 gene on each of the individual's two 5q13.2 chromosomes, with one of the SMN1 genes also being positive for the g.27134T>G SNP.

10. The method according to claim 6 further comprising:
counseling the individual of the individual's decreased risk of being a carrier (1+0) or silent carrier (2+0) of SMA based on the individual's copy number of the SMN1 gene and the presence or absence of the g.27134T>G SNP on one of the SMN1 genes.

11. The method according to claim 6 further comprising:
collecting the genomic DNA sample from the individual prior to the screening step.
